(19) United States
(12) Patent Application Publication
(10) Pub. No.: US 2026/0207657 A1
(43) Pub. Date: Jul. 23, 2026

(54) ANTISEPTIC COMPOSITIONS AND METHODS

(71) Applicant: Solventum Intellectual Properties Company, Maplewood, MN (US)

(72) Inventors: Ranjani V. Parthasarathy, Woodbury, MN (US); Kevin D. Landgrebe, Woodbury, MN (US); Katie F. Wlaschin, St. Paul, MN (US); Rajan B. Bodkhe, Woodbury, MN (US)

(73) Assignee: Solventum Intellectual Properties Company, Maplewood, MN (US)

(21) Appl. No.: 19/144,025

(22) PCT Filed: Nov. 30, 2023
(86) PCT No.: PCT/IB2023/062072

§ 371 Date: Jun. 27, 2025

Related U.S. Application Data
(60) Provisional application No. 63/477,466, filed on Dec. 28, 2022.

(51) Int. Cl. A61K 31/79; A61K 33/18; A61K 47/12; A61P 31/02

(52) U.S. Cl. CPC A61K 31/79; A61K 33/18; A61K 47/12; A61P 31/02

(57) ABSTRACT

An antiseptic composition includes an antimicrobial agent that includes molecular iodine (I₂), an iodide salt, a hydroxycarboxylic acid present at a concentration of at least 2 wt-%, based on the total weight of the antiseptic composition, and a nonionic surfactant. The antiseptic composition includes no amount of anionic surfactant.

### BACKGROUND

Antiseptic compositions have been discussed in, for example, U.S. Pat. Nos. 7,147,873, 6,838,078, 8,808,722, 10,052,384, U.S. Pat. Nos. 11,259,521, 11,096,958, and German Pat. DE10201401069.

### DETAILED DESCRIPTION

It is a standard practice in the industrialized world to disinfect the skin prior to any invasive procedure such as surgery, catheterization, or needle puncture to reduce the risk of infection. Decontamination of the oral cavity and nasal cavity also has been suggested to reduce the incidence of infection in cardiac surgery and/or to reduce spread of Methicillin Resistant Staphylococcus aureus (MRSA) in healthcare facilities. These products are often referred to as skin preps, nasal preps, oral preps, or simply “preps”.

Molecular iodine (I₂) has proven to be an outstanding antimicrobial agent for such antiseptic compositions. In many antiseptic compositions, iodine as antimicrobial agent is provided in solution in the form of an “iodophor” which is a complex of elemental iodine or triiodide with certain carriers. These iodophors function to not only increase the iodine solubility but to reduce the level of free molecular iodine in solution and to provide a type of sustained release reservoir of elemental iodine. Of available iodophors, povidone iodine is particularly useful.

Still other components have been shown to further boost the antimicrobial efficacy of molecular iodine. The addition of hydroxycarboxylic acids, for example, to antiseptic compositions as a buffer advantageously results in increased levels of bacterial “kill”. U.S. Pat. No. 7,147,873 (Scholz et al.) reports that elevated levels of hydroxycarboxylic acids (above 5% wt/wt) result in significantly improved antimicrobial efficacy. The use of hydroxycarboxylic acids in elevated levels in combination with iodophors like povidone iodine would thus seem rather desirable for most antiseptic compositions.

While desirable for higher microbial kill, elevated levels of certain hydoxycarboxylic acids shorten the effective life of the antimicrobial composition. The hydroxycarboxylic acid in certain cases tends to ultimately decrease the available free iodine and reduce the stability of the composition.

A variety of surfactants employed at high concentrations have been used to address the issue of composition instability. For examples, mixtures of anionic surfactants, zwitter-ionic surfactants, and non-ionic surfactants have been used to enhance stability. However, many of these surfactants, particularly anionic surfactants, present regulatory issues (U.S. FDA). Consequently, components that facilitate stability of these iodine and hydrocarboxylic acid containing compositions, and that do not present regulatory issues (e.g., are on the U.S. FDA list of approved inactive excipients) are desirable.

In this regard, surprisingly, it has been discovered that even in the absence of the aforementioned anionic surfactants, by increasing the concentration of certain non-ionic surfactants, stable compositions can be achieved. Further surprising, it has been discovered that such stable compositions can be achieved even at relatively low levels of total iodine (which provides benefits in terms of, for example, reducing skin irritation or blood level of iodine), where stability is more difficult to control.

The term “iodine stable” refers to a composition that does not suffer a loss of greater than about 25% wt/wt in the available iodine from the original value (i.e., concentration) when aged in a closed (i.e., sealed and does not allow evaporation of any of the components) and unreactive container at 40° C. for about 6 months.

The terms “tissue antiseptic composition,” “antiseptic composition,” “composition,” “skin prep,” and “prep” herein refer to a composition that is active against (i.e., effective at killing and/or deactivating) at least one species of bacteria on skin and/or mucosal tissue.

The term “available iodine” in the context of compositions of the present disclosure refers to all species of iodine (including triiodide ion and molecular iodine) that can be titrated by sodium thiosulfate.

The term “total iodine” in the context of the compositions of the present disclosure refers to the iodine content present in all forms, including iodine salts and molecular iodine (I₂).

The term “amine oxide” includes compounds, oligomers, and polymers comprising one or more amine oxide groups and compounds comprising a single amine oxide group.

As used herein, a material on the U.S. FDA list of approved inactive excipients refers to those materials listed in the inactive ingredients database on the U.S. FDA website (as of the 10/20/2022 update) for nasal or topical or oral use.

The terms “comprises” and variations thereof do not have a limiting meaning where these terms appear in the description and claims.

As used herein, “a,” “an,” “the,” “at least one,” and “one or more” are used interchangeably. Thus, for example, an antimicrobial composition that comprises “a” surfactant can be interpreted to mean that the antimicrobial composition includes “one or more” surfactants.

As used herein, the term “or” is generally employed in its sense including “and/or” unless the content clearly dictates otherwise.

The term “and/or” means one or all of the listed elements or a combination of any two or more of the listed elements (e.g., killing and/or inactivating a bacterium means inactivating, killing, or both inactivating and killing the bacterium).

Also herein, the recitations of numerical ranges by endpoints include all numbers subsumed within that range (e.g., 1 to 5 includes 1, 1.5, 2, 2.75, 3, 3.80, 4, 5, etc.).

The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

In some embodiments, the present disclosure is directed to an antiseptic composition that includes (i) an antimicrobial agent that includes molecular iodine (I₂) (e.g., in the form of an iodophor); (ii) an iodide salt (e.g., sodium iodide); (iii) a hydroxycarboxylic acid; and (iv) a non-ionic surfactant; and does not include appreciable amounts of an anionic surfactant.

In some embodiments, the antiseptic compositions may include an antimicrobial agent that include, or is in the form of, molecular iodine (I₂). In some embodiments, molecular iodine can be provided in the form of an iodophor. As in most iodine-containing patient preps, other iodine-containing species may be present in addition to iodine. Such species include, for example, hypoiodous acid (HOI), iodide (I⁻), triiodide (I₃⁻), iodate (IO₃⁻), and the like. It is widely recognized that molecular iodine is the most active antimicrobial species. See, for example, Disinfection, Sterilization, and Preservation by Seymour S. Block, 4^{th} edition, Chapter 8 “Iodine and Iodine Compounds,” Lea & Febiger, Philadelphia, PA., 1991. Minor amounts of Br⁻ and or Cl⁻ also may be present.

In most commercially available iodine disinfectants, in order to prevent rapid reduction of iodine to iodide the solutions are typically buffered to be slightly acidic (e.g., 6 or less, and often 2 to 6). Compositions that are too acidic can be irritating. The acidity is typically desired to maintain stability in the iodine solutions and to suppress conversion to other iodine species that are less germicidal. For example, commercial skin preps containing iodine generally have pH values in the range of 3 to 6, which favors stability of the molecular iodine species. HOI normally exists in very low levels relative to I₂ but has been reported as an effective antimicrobial and may contribute to kill in some compositions. IO₃⁻ is an effective oxidant only at pH values less than 4, where significant amounts of HIO₃ can exist.

As further background for understanding and practicing the present invention, molecular iodine is only slightly soluble in water (0.03 wt-% at 25° C.). Alkali metal iodides, which combine with iodine to form triiodide (I₃⁻), increase that solubility. Molecular iodine, however, can be very irritating at higher concentrations. For example, Lugol's solution (5% elemental iodine and 10% potassium iodide) and tincture of iodine (45% aqueous ethanol with 2% elemental iodine and 2.4% sodium iodide) have both been well documented to be quite irritating to the skin.

Many references have described the preparation of “iodophors,” which are complexes of elemental iodine or triiodide with certain carriers. These iodophors function to not only increase the iodine solubility but to reduce the level of free molecular iodine in solution and to provide a type of sustained release reservoir of elemental iodine. Iodophors are known using carriers of polymers such as polyvinylpyrrolidone, copolymers of N-vinyl lactams with other unsaturated monomers such as, but not limited to, acrylates and acrylamides, various polyether glycols including polyether-containing surfactants such as nonylphenolethoxylates and the like, polyvinyl alcohols, polycarboxylic acids such as polyacrylic acid, polyacrylamides, polysaccharides such as dextrose, and the like, and combinations thereof. A suitable group of iodophors includes polymers such as a polyvinylpyrrolidone (PVP), a copolymer of N-vinyl lactam, a polyether glycol (PEG), a polyvinyl alcohol, a polyacrylamide, a polysaccharide, and combinations thereof. Also reported in U.S. Pat. No. 4,597,975 (Woodward et al.) are protonated amine oxide surfactant-triiodide complexes that are also suitable iodophors for use in the present invention. Various combinations of iodophors can be used in the compositions of the present disclosure.

In some embodiments, the iodophor may be povidone-iodine. Suitable povidone-iodine can be obtained commercially as povidone-iodine USP, which is believed to be a complex of K30 polyvinylpyrrolidone, iodine, and iodide wherein the available iodine is present at 9 wt-% to 12 wt-%. As used herein, the “available iodine” for a composition may be determined by following the method in the United States Pharmacopeia Official Monographs for Povidone-Iodine, Assay for Available Iodine. Certain formulations may contain components that can interact with the method such as other anionic species. For this reason, the proper standards must be run to ensure accuracy, and solvent systems or reagents may need to be changed to ensure accuracy. One skilled in the art would appreciate these considerations.

In some embodiments, the iodophor may be present in the compositions at a concentration of at least 1 percent by weight (wt-%), at least 2.5 wt-%, at least 4 wt-%, or at least 5 wt-%, based on the total weight of the antiseptic composition. To prevent the dried composition from becoming excessively water soluble and/or to control irritation, iodine toxicity, and poor taste, the concentration of iodophor in the use composition may be present at not more than 15 wt-%, or not more than 10 wt-%, based on the total weight of the antiseptic composition.

Since iodophors may vary in the amount of available iodine it is usually more convenient to describe the concentration in terms of the available iodine level. In the present invention, whether from iodine or an iodophor or a combination thereof, the available iodine concentration may be at least 0.1 wt %, at least 0.2 wt-%, at least 0.25 wt-%, at least 0.4 wt-%, or at least 0.5 wt-%, based on the total weight of the antiseptic composition. Concentrations of available iodine below 0.1 wt-% may not be sufficiently bactericidal and may not lower microbial counts over longer periods of time (e.g., 12 hours or 24 hours). In some embodiments, the available iodine may be present at not more than 2 wt-%, not more than 1.5 wt-%, or not more than 1 wt-%, based on the total weight of the antiseptic composition. Concentrations of available iodine above 2 wt-% may be too irritating to wound and mucosal tissue and skin.

In some embodiments, iodide salts may be present in the antiseptic compositions of the present disclosure to facilitate stabilization of the iodine-containing solutions. Suitable iodide salts may include sodium iodide (NaI), potassium iodide (KI), calcium iodide (CaI₂), and zinc iodide (ZnI₂) and combinations thereof. In some embodiments, the iodide salt may be present at a total concentration of at least 0.25 wt-%, at least 0.5 wt-%, at least 1 wt-%, at least 1.5 wt-%, at least 2.0 wt-%, or at least 2.5 wt-%, based on the total weight of the antiseptic composition. In certain embodiments, the iodide salt may be present in the antiseptic composition at a total concentration of no greater than 10 wt-%, or no greater than 5 wt-%, or no greater than 3% based on the total weight of the antiseptic composition. Compositions including less than a total concentration of about 0.25 wt-% iodide salt may not be iodine stable.

In some embodiments, the total iodine present in the compositions of the present disclosure may be
0.35 to 3 wt-%, 0.8 to 2.7 wt-%, or 0.8 to 1.5 wt-%, based on the total weight of the composition. Generally, compositions having lower total iodine levels (e.g., less than about 1.5 wt-%) while being more difficult to stabilize, are less likely to cause irritation to the user.

In some embodiments, the compositions of the present disclosure may be buffered to prevent pH drift during storage. For example, the iodine-containing systems may be maintained at a pH of 2 to 6, or 3 to 5, or 3 to 3.8. As the pH is raised above 6, the iodine can be rapidly converted to iodide, thus inactivating the antimicrobial effectiveness. The composition may become irritating if the pH falls below 2. In this regard, in some embodiments, in the compositions of the present disclosure, the pH may be adjusted to 3.0 to 4.5. While conventional compositions have included a variety of organic and inorganic buffers at concentrations of 0.1 wt-% to 2 wt-%, in some embodiments, the compositions of the present disclosure may include certain hydroxycarboxylic acid buffers that can be used in much higher buffer concentrations. In some embodiments, a hydroxycarboxylic acid buffer may be present in an amount of greater than 1 wt-%, greater than 2.5 wt-%, greater than 3 wt-%, greater than 5 wt-%, or greater than 6 wt-%, based on the total weight of the antiseptic composition. These compositions (i.e., with a pH adjusted to 3.0 to 3.8 and a relatively high hydroxycarboxylic acid buffer concentration) are substantially nonirritating to tissue (e.g., skin and mucosal tissue).

In some embodiments, this level of buffer may be desirable for antiseptic compositions that include povidone-iodine (particularly povidone-iodine USP) as the antimicrobial agent. In these systems, the level of rapid microbial kill increases significantly and for some systems in a linear fashion with the molar concentration of the hydroxycarboxylic acid.

In some embodiments, suitable hydroxycarboxylic acid buffers may include one or more compounds disclosed in U.S. Pat. No. 7,147,873 (Scholz et al.) (which is incorporated herein by reference in its entirety) and are represented by the formula: R¹(CR²OH)ₙ(CH₂)ₘCOOH wherein: R¹ and R² are each independently H or a (C1-C8) alkyl group (saturated straight, branched, or cyclic group), a (C6-C12) aryl, or a (C6-C12) aralkyl or alkaryl group (saturated straight, branched, or cyclic alkyl group), wherein R¹ and R² may be optionally substituted with one or more carboxylic acid groups; m=0 or 1; and n=1-3 or n=1-2.

In some embodiments, the buffers and other excipients that contain hydrocarbon groups are saturated or contain low levels of unsaturation to prevent iodine addition, which may deplete the iodine in the composition and/or produce toxic species. In some embodiments, the level of unsaturation in the composition is no greater than 50 milliequivalents per liter (meq/L), no greater than 5 meq/L, or no greater than 0.5 meq/L unsaturation.

In some embodiments, suitable hydroxycarboxylic acid buffers may include beta- and alpha-hydroxy acids (BHAs, AHAs, respectively, collectively referred to as hydroxy acids (HAs)), salts thereof, lactones thereof, and/or derivatives thereof (preferably, alpha-hydroxy acids are used). These may include mono-, di-, and tri-functional carboxylic acids. Suitable HAs may have 1 or 2 hydroxyl groups and 1 or 2 carboxylic acid groups. Suitable HAs include, but are not limited to, lactic acid, malic acid, citric acid, 2-hydroxybutanoic acid, 3-hydroxybutanoic acid, mandelic acid, gluconic acid, tartaric acid, salicylic acid, as well as derivatives thereof (e.g., compounds substituted with hydroxyls, phenyl groups, hydroxyphenyl groups, alkyl groups, halogens, as well as combinations thereof). Suitable HAs include lactic acid, malic acid, and citric acid. These acids may be in D, L, or DL form and may be present as free acid, lactone, or salts thereof. Other suitable HAs are described in U.S. Pat. No. 5,665,776 (Yu et al.). In some embodiments, suitable HAs for use with iodine, and in particular with povidone-iodine, are lactic and malic acid and combinations thereof. Various combinations of hydroxycarboxylic acids can be used if desired.

It has been discovered that monocarboxylic acids (e.g., lactic acid) may have superior stability and good viability while still being efficacious in formulations without anionic surfactants. Addition of di and trifunctional carboxylic acids may improve efficacy but such improvement may be at the expense of chemical degradation and decreased viability (especially when used repeatedly).

In some embodiments, a hydroxycarboxylic acid buffer may be present in a molar concentration of at least 0.1 molar, at least 0.3 molar, at least 0.45 molar, or at least 0.6 molar. For formulations where very rapid microbial kill on skin is desired, the hydroxycarboxylic acid concentration may be in excess of 0.7 molar.

It is believed that the antimicrobial efficacy of antimicrobial compositions containing povidone-iodine is directly related to the molar concentration of hydroxycarboxylic acid buffer. With sufficiently high levels of hydroxycarboxylic acid buffer, compositions containing povidone-iodine are able to reduce the normal skin flora on a dry human skin site (typically, the back or abdomen) by an average of greater than or equal to 2 logs in only 2 minutes following a 30-second scrub, and following a simple painting application (no scrubbing) where the site is painted 3 times when tested according to ASTM testing method E1173-93.

In some embodiments, the concentration of hydroxycarboxylic acid buffer in weight percent of the ready to use composition (i.e., a composition that need not undergo additional dilution before application to tissue) may be at least 2% by weight, at least 5 wt-%, or at least 7 wt-%, based on the total weight of the composition. The concentration of hydroxycarboxylic acid buffer may be no greater than about 15 wt-%, or no greater than about 10 wt-%, based on the weight of the composition. It may also be convenient in some applications to supply concentrates that have much higher concentration of hydroxycarboxylic acid buffer but when diluted to the use concentration fall within the specified ranges.

For effective kill on skin and mucosal tissue, the compositions of the present disclosure may include one or more surfactants. The surfactants may be compatible with the antimicrobial agent, the iodide salts, the hydroxycarboxylic acid buffer, as well as any other components of the composition. It may be desirable when formulating with a film-forming polymer to include one or more surfactants to enhance solubility and stability of the polymer in the composition. In addition, surfactants may help the compositions to wet the skin and ensure a smooth uniform coating. It may be desirable to provide a coating that has complete coverage to ensure easy error-free application. On tissues that are hard to visualize, such as most mucosal surfaces, it may be desirable to use surfactants to help wetting and to ensure the antimicrobial agent will be distributed by diffusion and or capillary action across the tissue. On skin, a thin, relatively uniform coating may be applied that will dry rapidly. In addition, certain surfactants may increase the antimicrobial activity.

In some embodiments, suitable surfactants for use in the compositions of the present disclosure include nonionic surfactants. It has been found that polyalkoxylated, and in particular polyethoxylated, nonionic surfactants can stabilize film-forming polymers in aqueous solutions particularly well. In general, useful polyalkoxylated nonionic surfactants may have a hydrophile/lipophile balance (HLB) of at least 12, at least 14, or at least 16. Useful polyalkoxylated nonionic surfactants may have an HLB of no greater than 20. When using combinations of nonionic surfactants, a weight average HLB is used to determine the HLB of the nonionic surfactant system. As used herein, the HLB is defined as one-fifth the weight percentage of ethylene oxide segments in the surfactant molecule.

### Nonionic

Surfactants of the nonionic type that may be useful include:
1. Polyalkoxylated alkanols. Surfactants such as those commercially available under the trade designation BRIJ from ICI Specialty Chemicals, Wilmington, DE, having an HLB of at least 14 have proven useful. In particular, BRIJ 78 and BRIJ 700, which are stearyl alcohol ethoxylates having 20 and 100 moles of polyethylene oxide, respectively, have proven very useful. Also useful is a ceteareth 55, which is commercially available under the trade designation PLURAFAC A-39 from BASF Corp., Performance Chemicals Div., Mt. Olive, NJ. Other surfactants could include Steareth-100 (Hetoxyl STA-100), Steareth-40 (Ethal SA-40), Ceteareth-25 (C-25) sold by Ethox Chemicals. Greenville, SC.
2. Polyalkoxylated alkylphenols. Useful surfactants of this type include polyethoxylated octyl or nonyl phenols having HLB values of at least 14, which are commercially available under the trade designations ICONOL and TRITON, from BASF Corp., Performance Chemicals Div., Mt. Olive, NJ and Union Carbide Corp., Danbury, CN, respectively. Examples include TRITON X100 (an octyl phenol having 15 moles of ethylene oxide available from Union Carbide Corp., Danbury, CN) and ICONOL NP70 and NP40 (nonyl phenol having 40 and 70 moles of ethylene oxide units, respectively, available from BASF Corp., Performance Chemicals Div., Mt. Olive, NJ). Sulfated and phosphated derivatives of these surfactants are also useful. Examples of such derivatives include ammonium nonoxynol-4-sulfate, which is commercially available under the trade designation RHODAPEX CO-436 from Rhodia, Dayton, NJ.
3. Polyethylene Oxides. Surfactants based on polymers of ethylene oxide (EO) may be useful. Polyethylene oxides of molecular weights greater than 300 could also be used
4. Polaxamers. Surfactants based on block copolymers of ethylene oxide (EO) and propylene oxide (PO) have been shown to be effective at stabilizing film-forming polymers and provide good wetting. Both EO-PO-EO blocks and PO-EO-PO blocks are expected to work well as long as the HLB is at least 14, and preferably at least 16. Such surfactants are commercially available under the trade designations PLURONIC and TETRONIC from BASF Corp., Performance Chemicals Div., Mt. Olive, NJ. It is noted that the PLURONIC surfactants from BASF have reported HLB values that are calculated differently than described above. In such situation, the HLB values reported by BASF should be used. For example, preferred PLURONIC surfactants are L-64 and F-127, which have HLBs of 15 and 22, respectively. Although the PLURONIC surfactants are quite effective at stabilizing the compositions of the present invention and are quite effective with iodine as the active agent, they may reduce the antimicrobial activity of compositions using povidone-iodine as the active agent.
5. Polyalkoxylated esters. Polyalkoxylated glycols such as ethylene glycol, propylene glycol, glycerol, and the like may be partially or completely esterified, i.e., one or more alcohols may be esterified, with a (C8-C22)alkyl carboxylic acid. Such polyethoxylated esters having an HLB of at least 14, and preferably at least 16, are suitable for use in compositions of the present invention.
6. Alkyl Polyglucosides. Alkyl polyglucosides, such as those described in U.S. Pat. No. 5,951,993 (Scholz et al.), starting at column 9, line 44, are compatible with film-forming polymers and may contribute to polymer stability. Examples include glucopon 425, which has a (C8-C16)alkyl chain length with an average chain length of 10.3 carbons and 1-4 glucose units.
7. Polyethylene oxide extended sorbitan monoalkylates (i.e., POLYSORBATES). In particular, a useful surfactant of this type could be Polysorbate 20 commercially available as NIKKOL TL-10 (from Barret Products)

### Zwitterionic

In some embodiments, suitable surfactants for use in the compositions of the present disclosure may optionally include zwitterionic surfactants. Surfactants of the zwitterionic type include surfactants having tertiary amine groups which may be protonated as well as quaternary amine-containing zwitterionic surfactants and that are on the U.S. FDAs list of inactive excipients. Suitable zwitterionic surfactants may include:
1. Amine oxides. This class of surfactants can be represented by the following formula:

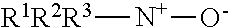

Wherein at least one of R¹, R², and R³ is a (C8-C22) alkyl group. Preferably, the amine oxide is that commercially available as MACKAM LO (R¹ is a C12 alkyl group and R¹ and R² are hydrogen) from McIntyre Group Ltd., University Park, IL.
Amine oxides have been reported to complex molecular iodine and/or triiodide, thus stabilizing some iodine solutions. In this regard, in some embodiments, the compositions of the present disclosure may optionally include one or more amine oxides. The amine oxides may be a compound, oligomer, or polymer comprising one or more amine oxide groups or compound comprising a single amine oxide group. Suitable amine oxides are acceptable for a topical pharmaceutical formulation, i.e., one that is not appreciably irritating or toxic to skin or mucosal tissue. Examples of suitable amine oxides for use in the present compositions are those having surface activity (surfactants) and are generally characterized as having at least one alkyl group having a weight average chain length of greater than 6 carbon atoms, greater than 8 carbon atoms, or greater than 10 carbon atoms. Also included are amine oxides of aromatic tertiary amines and tertiary amines comprising an alkaryl group such as dimethylbenzylamine oxide. The trialkylamine oxides (e.g., lauryldimethylamine oxide) have at least one C6-C22 alkyl group wherein the alkyl group or groups can be optionally substituted in or on the chain by N, O, or S. The alkaryldialkyl amine oxides have at least one alkaryl group having at least 7 carbon atoms and alkyl groups of C1-C22 optionally substituted in or on the chain by N, O, or S. Particularly useful are myristamine oxides such as myristamidopropyldimethylamine oxide, lauramine oxides such as lauramidopropyldialkylamine oxide, dihydroxyethyldodecylamine oxide, and mixtures thereof. Additional amine oxides suitable for use in the compositions of the present disclosure include those commercially available under the trade designations AMMONYX LO, LMDO, and CO, which are lauryldimethylamine oxide, laurylamidopropyldimethylamine oxide, and cetyl amine oxide, all from Stepan Co. (Northfield, IL), as well as N-oxides of ethoxylated tertiary amines such as those having the structure R₂—N(AO)₂, R—N(AO)₂ or R—N(AO)—R′N(AO)₂ or R—N(AO)—R′N(AO) where AO=polyalklylene oxide having 1-100 moles of ethylene oxide or propylene oxide residues and R=C1-C22, preferably C8-C18 aliphatic hydrocarbon optionally substituted with N, O, or S. R′ is a connecting group and is most preferably a C2-C10 alkylene group. Examples include Ethomeen and alkoxylated Duomeen tertiary amines available from Akzo Nobel, Additional examples of suitable tertiary amine oxides include: N, N-dimethyl benzylamine oxide; N-ethyl, N-lauryl benzylamine oxide; N-methyl N-ethyl benzylamine oxide; 4-alkyl pyridine N-oxide, 3-alkyl pyridine N-oxide, 2 alkyl pyridine N-oxide; alkyl pyrazine N-oxide; alkyl pyrazine N, N′-dioxide; N-alkyl piperidine N-oxide; N, N′-alkyl piperazine N-oxide; N, N′ diakyl piperazine N, N′-dioxide; N-alkyl morpholine N-oxide; alkyl substituted quinoline N-oxide; N, N-dialkyl cyclohexylamine N-oxide; N, N-dialkylaniline N-oxide, and the like as well as mixtures thereof.
In some embodiments, the amine oxides in the antiseptic compositions are present in an amount such that the ratio of the total moles of amine oxide to total moles of available iodine is less than about 2. In some embodiments, the total amine oxide present in the compositions of the present disclosure may be at least 0.25 wt-%, at least 0.50 wt-%, or at least 1 wt-%, based on the total weight of the antiseptic composition.
In some embodiments, amine oxides, if present, are on the U.S. FDA's list of approved inactive excipients.
2. Ammonium Carboxylate Zwitterionics. This class of surfactants can be represented by the following formula:

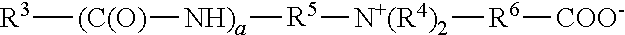

wherein: a=0 or 1; R³ is a (C7-C21)alkyl group (saturated straight, branched, or cyclic group), a (C6-C22)aryl group, or a (C6-C22)aralkyl or alkaryl group (saturated straight, branched, or cyclic alkyl group), wherein R³ may be optionally substituted with one or more N, O, or S atoms, or one or more hydroxyl, carboxyl, amide, or amine groups; R⁴ is H or a (C1-C8)alkyl group (saturated straight, branched, or cyclic group), wherein R⁴ may be optionally substituted with one or more N, O, or S atoms, or one or more hydroxyl, carboxyl, amine groups, a (C6-C9)aryl group, or a (C6-C9)aralkyl or alkaryl group; and R⁵ and R⁶ are each independently a (C1-C10)alkylene group that may be the same or different and may be optionally substituted with one or more N, O, or S atoms, or one or more hydroxyl or amine groups.

In some embodiments, in the formula above for ammonium carboxylate zwitterionics, R³ is a (C1-C16)alkyl group, R⁴ is a (C1-C2)alkyl group preferably substituted with a methyl or benzyl group. When R⁴ is H it is understood that the surfactant at higher pH values could exist as a tertiary amine with a cationic counterion such as Na, K, Li, or a quaternary amine group.

Examples of such zwitterionic surfactants include, but are not limited to: certain betaines such as cocobetaine, and cocamidopropyl betaine (commercially available under the trade designations MACKAM LO, MACKAM CB-35, and MACKAM L from McIntyre Group Ltd., University Park, IL); monoacetates such as sodium lauroamphoacetate; diacetates such as disodium lauroamphoacetate; amino- and alkylamino-propionates such as lauraminopropionic acid (commercially available under the trade designations MACKAM 1L, MACKAM 2L, and MACKAM 151L, respectively, from McIntyre Group Ltd.).
3. Ammonium Sulfonate Zwitterionics. This class of zwitterionic surfactants are often referred to as “sultaines” or “sulfobetaines” and can be represented by the following formula

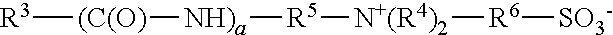

wherein R³—R⁶ and “a” are defined as above for ammonium carboxylate zwitterionics. Examples include cocamidopropylhydroxysultaine and lauramidopropylhydroxy sultaine (commercially available as MACKAM 50-SB from McIntyre Group Ltd.).
4. Phospholipid Zwitterionics. These surfactants are characterized as having at least one anionic phosphate group, one cationic ammonium group (either protonated or quaternary), and at least one alkyl, alkenyl, aralkyl, or aralkenyl group of at least 8 carbon atoms. Many surfactants of this class of surfactants can be represented by the following formula:

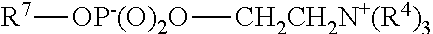

wherein R⁴ is-denned above or ammonium carboxylate zwitterionics and R⁷ is R³ (as defined above for ammonium carboxylate zwitterionics) with the proviso that R⁷ also may comprise multiple R³ groups as would be the case if R⁷ were a glycerol ester derivative as, for example, in phosphatidylcholine. Examples include lecithins, phosphatidylcholine and phosphatidylethanol amine. The so called “reverse phospholipids” which possess a quaternary ammonium group in the chain and a terminal phosphate group are also possible such as those sold by Unigema/Croda under the tradename Arlasilk Phospholipid CDM (coco PG-dimonium chloride phosphate), Arlasilk Phospholipid EFA (Linoleamidopropyl PG-Dimonium Chloride Phosphate), and the like.

In some embodiments, the compositions may be free of (or substantially free of) anionic surfactants. As discussed above, while useful in stabilizing iodine and hydrocarboxylic acid containing compositions, known anionic surfactants present undesirable regulatory issues. In this regard, in some embodiments, the compositions of the present disclosure may include anionic surfactants in an amount of less than 1 wt. %, less than 0.5 wt. %, less than 0.1 wt. %, less than 0.01 wt. %, or 0 wt. %, based on the total weight of the composition; or may include surfactants not on the U.S. FDA's list of approved inactive excipients in an amount of less than 1 wt. %, less than 0.5 wt. %, less than 0.1 wt. %, less than 0.01 wt. %, or 0 wt. %, based on the total weight of the composition

In some embodiments, surfactants (e.g., nonionic surfactants and optionally zwitterioinic surfactants) may be present in the antiseptic compositions of the present disclosure in a total amount of at least 0.25 wt %, at least 0.5 wt-%, or at least 1 wt-%, based on the total weight of the composition. In some embodiments, surfactants may be present in the compositions in a total amount of no greater than 10 wt-%, no greater than 7 wt-%, no greater than 5 wt-%, or no greater than about 3 wt-%, based on the total weight of the composition. For embodiments in which zwitterionic surfactant such as lauramine oxide is added, lower levels of non-ionic surfactant may be needed to keep the formulation from degrading (i.e., adding high levels of lauramine oxide (0.75%) can result in greater chemical degradation. Too little surfactant may result in an unstable composition (especially upon exposure to elevated temperatures) and/or reduced antimicrobial efficacy on tissue. Too much surfactant can undermine the substantivity of the dried composition on skin and contribute to tissue irritation.

In some embodiments, when the composition comprises povidone iodine, certain types of non-ionic surfactants may be employed based on the concentration of povidone iodine. Specifically, lower HLB and higher hydroxyl value non-ionic surfactants like ceteareth-25 may provide the greatest physical stability when the povidone iodine concentration is relatively high and higher HLB and lower hydroxyl value surfactants such as steareth-100 may provide the greatest physical stability when used with relatively lower levels of povidone iodine. In this regard, in some embodiments, when the povidone iodine concentration is greater than 3 wt. %, greater than 4 wt. % or greater than 5 wt. %, based on the total weight to the composition, the non-ionic surfactant may have an HLB value of between 10 and 17, between 12 and 17, or between 16 and 17 and a hydroxyl value (—OH value (mg KOH/g)) of between 30 and 50, between 35 and 45, or between 40 and 45. In some embodiments, the when the povidone iodine concentration is less than 3 wt. % or less than 2.5 wt. %, based on the total weight to the composition, the non-ionic surfactant may have an HLB value of between 17 and 20, between 18 and 20, or between 18 and 19 and a hydroxyl value (—OH value (mg KOH/g)) of between 10 and 25 or between 12 and 20, or between 12 and 15.

In some embodiments, nonionic surfactants may be present in the antiseptic compositions of the present disclosure in an amount of between 0.25 wt % and 10 wt-%, between 0.5 wt-% and 7 wt-%, or between 0.5 wt-% and 3 wt-%, based on the total weight of the composition. It is surprising that with these relatively high concentrations of nonionic surfactants, the iodine containing compositions can be stabilized the in the absence of charged surfactants (and even in the presence of charged species/charged complexes such as anionic iodide and cationic thickeners).

In some embodiments, the compositions of the present disclosure may include monosaccharides or sugar alcohols. Suitable monosaccharides for use in the compositions described herein have the chemical formula (CH₂O)ₙ₊ₘ with the chemical structure H(CHOH)ₙC═O(CHOH)ₘH. If n or m is zero, it is an aldehyde and is termed an aldose, otherwise it is a ketone and is termed a ketose. Monosaccharides contain either a ketone or aldehyde functional group, and hydroxyl groups on most or all of the non-carbonyl carbon atoms. The monosaccarides may be 5 or 6 carbon atom (n+m=5 or 6) compounds. They may be found in the D or L form or a combination thereof. The most preferred monosaccharides are xylose, xylulose, lyxose, mannose, maltose, sorbose, erythrose, glucose (dextrose), fructose, galactose, and ribose

The term “sugar alcohol” is understood to mean a monosaccharide or a disaccharide in which the aldehyde group of the first carbon atom is reduced to a primary alcohol. They include the following sugar alcohols: xylitol, sorbitol, mannitol, maltitol, erythritol, lactitol and arabitol or combinations thereof. Sugar alcohols may be those derived from monosaccharides (i.e., alcohols of a monosaccharide) including xylitol, mannitol, or combinations thereof. The sugar alcohol may be xylitol. As used herein, the term “alcohol of a monosaccharide” is understood to mean a monosaccharide in which the aldehyde group of the first carbon atom is reduced to a primary alcohol.

These monosaccharides and/or sugar alcohols have been found to further increase the efficacy (i.e., speed and/or extent of bacterial kill) of iodine containing compositions. (See e.g., US Application Publication No. 2009/0169647 (Scholz)).

The monosaccharides and/or sugar alcohols may be present in a concentration of at least 0.25 wt-%, at least 0.5 wt-%, at least 1 wt-%, at least 2 wt-%, at least 4 wt-%, or at least 5 wt-%, based on the total weight of the composition. The concentration may be adjusted to ensure improved antimicrobial performance and/or to improve the taste of the composition if it is applied to the oral cavity, esophageal cavity, nasal passages, or anterior nares. The upper limit may be determined by the solubility limit of the monosaccharide and/or sugar alcohol.

In some embodiments, it may be desirable to add one or more thickening agents, such as polymeric thickeners (which may be film-forming polymers), and/or film-forming polymers, to the antiseptic compositions to improve substantivity (e.g., resistance to wash off by mucus secretions in the nose, blood and body fluid exposure), improve adhesion of PSA-coated products, increase viscosity to prevent dripping, etc., reduce the tack of the compositions, and/or reduce the loss of available iodine during storage. Suitable polymeric thickeners and/or film-forming polymers of the antiseptic compositions of the present disclosure are substantive and resist removal by prolonged exposure to fluids such as water, saline, and body fluids, yet can be easily and gently removed without the need for organic solvents.

Certain skin antiseptic compositions of the present disclosure resist removal particularly well after they are dry. These compositions generally have lower viscosity (e.g., less than 1000 cps), and preferably greater than 10 cps, and have polymers with generally lower molecular weight (e.g., less than 200,000 daltons).

Antiseptic compositions for use on wound and mucosal tissues such as in the nose and anterior nares, however, may have a higher viscosity in order to retain the composition on the tissue (which is often wet) longer and to prevent dripping and mess. These compositions may have a viscosity greater than 100 cps, in greater than 500 cps, greater than 1000 cps, greater than 2000 cps, greater than 5000 cps, or greater than 8000 cps. These compositions may be thickened with one or more of the following:
a. Polymeric thickeners
b. Hydrophobically modified polymeric thickeners
c. Polymer/surfactant combinations
d. Emulsifiers (including waxes)
e. Inorganic colloidal thickeners

In some embodiments, suitable polymeric thickeners (which may be film-formers) may have both hydrophilic and hydrophobic moieties. The polymeric thickeners may include relatively high levels of total hydrophobic monomers. The polymers may be relatively hydrophobic to provide good substantivity and prolonged adhesion of PSA-coated products. The polymers may be formed using a hydrophobic monomer level of at least 50 wt-%, and often as high as 80 wt-%, based on the total weight of the polymerizable composition (or, based on the total weight of the polymer). Various combinations of hydrophobic monomers can be used if desired.

The polymeric thickeners (which may be film-forming polymers) can be nonionic, anionic, cationic, or zwitterionic. In some embodiments, the polymer thickeners may be cationic, as such thickeners were, surprisingly, found to be useful in forming stable, persistent compositions. Specifically, cationic thickeners allowed for viscous formulations (1000 cp-15000 cp) that were resistant to wash-off and were able to remain in place for efficacy over longer periods of time for decolonization of skin and mucosal surfaces.

In certain embodiments, the polymeric thickeners may be cationic polymers, such as those that include side-chain functional amine groups, which can be film-forming polymers. Examples of such groups include protonated tertiary amines, quaternary amines, amine oxides, and combinations thereof.

In some embodiments, polymeric thickeners may be vinyl polymers prepared from amine group-containing monomers. The vinyl polymers may have a Tg of at least 30° C., or at least 50° C. One method of measuring the Tg of a polymer may involve the utilization of a Differential Scanning Calorimeter (DSC, e.g., the PYRIS 7-Series Thermal Analyzer, Perkin-Elmer, Shelton, CN) in the range of −100° C. to +100° C. at a rate of 20° C. per minute.

For certain polymeric thickeners, the amine group-containing monomers can be used to prepare the polymers in an amount of at least 15 wt-%, at least 20 wt-%, even at least 25 wt-%, or at least 30 wt-%, based on the total weight of the polymerizable composition (and preferably, based on the total weight of the polymer). The equivalent weight of the amine group contained in the polymer may be at least 300, at least 350, at least 400, or at least 500, grams polymer per equivalent of amine group. The equivalent weight of the amine group contained in the polymer may be no greater than 3000, no greater than 1500, no greater than 1200, or no greater than 950, grams polymer per equivalent of amine group.

Examples of polymeric thickeners that are film-forming polymers and that are PSAs at room temperature include those based on side-chain functional amine group monomers in combination with long chain alkyl acrylic polymers and optionally other hydrophilic monomers.

In certain embodiments, one or more polymeric thickeners and/or film-forming polymers (preferably substantive film-forming polymeric thickeners), may be present in the antiseptic composition in a total amount of at least 2 wt-%, at least 3 wt-%, or at least 5 wt-%, based on the total weight of antiseptic composition. In certain embodiments, one or more polymeric thickeners and/or film-forming polymers (preferably substantive film-forming polymeric thickeners), may be present in the antiseptic composition in a total amount of no greater than 10 wt-%, or no greater than 8 wt-%, based on the total weight of antiseptic composition.

It may be desirable to include one or more other (secondary) antimicrobial agents as preservatives and/or active ingredients in combination with iodine. Other active ingredients can include cationics such as polyhexamethylene biguanide (PHMB, COSMOCIL CQ from Arch Biocides), chlorhexidine salts such as chlorhexidine gluconate, chlorhexidine acetate, benzalkonium chloride, benzethonium chloride, methylbenzethonium chloride, octenidine, cetyl pyridinium chloride, and the like, as well as other cationic antiseptics disclosed in U.S. Patent Application Publication No. 2006/0051384. Natural oil antiseptics such as those disclosed in U.S. Patent Application Publication No. 2006/0051384 may be added. In addition, it may be desirable to add antimicrobial lipids such as those described in U.S. Patent Application Publication No. 2005/0089539, although in certain embodiments, compositions of the present invention do not include antimicrobial lipids. Phenolic type antimicrobials also may be useful such as triclosan, parachlorometaxylenol and others disclosed in U.S. Patent Application Publication No. 2006/0052452.

It also may be desirable to add preservatives such as methyl, ethyl, propyl, and butyl paraben, 2 phenoxyethanol, hydantoins, diazolidinyl urea, and the like.

Compositions of the present disclosure may additionally employ adjunct components conventionally found in pharmaceutical compositions in their art-established fashion and at their art-established levels. Thus, for example, the compositions may contain additional compatible pharmaceutically active materials for combination therapy (such as supplementary antimicrobials, anti-parasitic agents, antipruritics, astringents, local anaesthetics, or anti-inflammatory agents), or may contain materials useful in physically formulating various dosage forms of the present invention, such as excipients, dyes, perfumes, lubricants, stabilizers, skin penetration enhancers, preservatives, or antioxidants, flavorants, flavor masking agents, odor masking agents, anti-inflammatories, antioxidants, vitamins, enzymes, enzyme inhibitors, growth factors, and sensates to induce a cool or warm feeling such as menthol, and the like.

In some embodiments, the antiseptic compositions of the present disclosure may have one or more of the following properties: relatively high levels of bacterial kill; relatively rapid speed and/or length of bactericidal activity; not likely to generate bacterial resistance; capable of delivering iodine (I2) over a period of time; suitable for use on sensitive tissues such as mucosal tissue including vaginal, oral, esophageal and nasal tissue; relatively non-irritating to a majority of patients; acceptable odor; acceptable taste in the event some of the composition is deliberately used in the oral or esophageal cavity or if the composition is placed in the nose and migrates up the nasal passages and down the throat; good adhesion to the skin and/or mucosal tissue when both wet and dry; sufficiently high viscosity to provide substantivity to mucosal tissue such that the residence time in the nose or other mucosal tissue (e.g., oral, vaginal, or esophageal) is increased over a non-thickened formulation; preferably good adhesion of pressure sensitive adhesive (PSA) coated products such as incise drapes, tapes, wound dressings, and the like, over the dried prep on skin (preferably, for long periods of time, e.g., hours to days); resist lift off of PSA-coated products over the dried prep on skin while under stress as typically occurs during retraction in surgery; can be removed relatively easily, preferably without the need for organic solvent-based removers and does not lose more than 20% wt/wt iodine (and preferably not more than 10% wt/wt iodine) over the shelf life of the product.

In some embodiments, the antiseptic compositions of the present disclosure possess many or all of the above-mentioned characteristics. Significantly, they may provide rapid broad spectrum microbial kill (bacteria, viruses, mold, yeast etc.), with very little or no chance of microbial resistance, are well tolerated on mucosal tissue, and have an acceptable odor and taste. Furthermore, they may be gentle to tissue and can be removed with a water-soaked or soap and water-soaked fabric, such as a towel or simple gauze.

In some embodiments, it may be useful to have a solution that is not thickened so it can be used as a spray for easy application. These compositions that do not have any thickener may be useful especially if they have similar efficacy and can reduce microbial counts for prolonged periods of time on tissue compared to that of a thickened composition.

In some embodiments, the compositions may be iodine stable at relatively high iodine concentrations used in the “ready to use” composition, in that they feature more available iodine (and thus more antimicrobial efficacy) after significant periods of storage than certain other iodophor containing antimicrobial compositions that include elevated levels of hydroxycarboxylic acids. Certain embodiments do not suffer a loss of greater than about 15%, greater than about 10%, or greater than about 5%, in the available iodine from the original weight percentage in the composition when aged in a closed and unreactive container at 40° C. for about 6 months. As used herein “ready to use” refers to the composition intended to be applied (e.g., to skin or mucosal tissue) without dilution.

In some embodiments, the compositions of the present disclosure are also physically stable, in that they show no indicia of visible gross phase separation (e.g., precipitation, phase split, settling) after storage at about 50° C. for 4-7 days. While certain embodied compositions may become separated into two parts with differential viscosities, sometimes, the lower part appears to be gelatinous, the absence of gross precipitation and/or settling means these samples may be considered physically stable. Other compositions of the present disclosure show no visible changes. Also, certain compositions of this disclosure may be thickened with emulsifier systems that form emulsions and dispersions. These systems are considered physically stable if there is no syneresis and if the viscosity does not drop to less than half the initial viscosity when aged for 30 days at 40° C. and allowed to equilibrate at 23° C. for 24 hours.

In some embodiments, samples tested after the freeze-thaw test (as set forth in the Examples), can also separate into two parts with differential viscosities (sometimes, the lower part appears to be gelatinous).

In some embodiments, compositions of the present disclosure are also generally substantive, and may be substantive even while in moist environments, such as the nose, anterior nares, and vaginal vault and remain on any of these tissues for longer periods of time than typical antiseptics such as BETADINE 10% povidone-iodine solution (Purdue Frederick, Norwalk, CN).

A “substantive” composition is one that when placed in the anterior nares has visible iodine still present 30 minutes (min) in a majority of subjects after instillation of 0.25 milliliter (mL) with a cotton bud and gently massaging the nostrils for 30 seconds to ensure an even distribution (as long as the patient does not discharge or deliberately or inadvertently wipe the product away). In some embodiments, substantive compositions remain present in the anterior nares for 45 min, or for 60 min, post instillation. This is conveniently determined by dabbing the inside of the anterior nares with a white tissue such as a KLEENEX tissue or by imparting color to the composition (e.g., inclusion of a small amount of a dye or a colored active such as povidone-iodine in sufficient concentration that a relatively dark color results on the skin that can be easily seen as present or not).

Many of the compositions of this disclosure may also be “skin substantive” and thus a composition applied and allowed to dry resists removal from skin for at least 15 seconds when tested as described in the “Substantivity Test” described in U.S. Pat. No. 7,147,873 (Scholz et al). For use on skin, the compositions may be even more substantive and resist being removed under the same conditions for at least 30 seconds, at least 45 seconds, or at least 60 seconds. This is conveniently determined by imparting color to the composition (e.g., inclusion of a small amount of a dye or a colored active such as povidone-iodine in sufficient concentration that a relatively dark color results on the skin that can be easily seen as present or not).

The dried films of certain antiseptic compositions of the present disclosure that include a film-forming polymer are generally flexible and durable. That is, they do not crack or flake off as brittle films might do. Significantly, film-forming polymers contribute to achieving a delicate balance between low tack and flexibility.

Although antiseptic compositions of the present disclosure can be of a wide variety of viscosities, in some embodiments, compositions possess viscosities that ensure the formulations go on easily and form a substantive film, particularly on wet tissue (such as mucosal tissue). The Brookfield viscosity of the compositions may be greater than 100 Centipoise (cps), greater than 500 cps, greater than 1000 cps, greater than 2000 cps, or greater than 5000 cps. Certain skin antiseptic compositions of the present disclosure may resist removal particularly well after they are dry. These compositions generally have lower viscosity (e.g., less than 1000 cps). Viscosities herein are measured at 23° C. using a Brookfield LVT viscometer and the procedure described in the Examples Section.

A relatively low viscosity ensures that the composition can be painted on the skin or mucosal tissue with little effort in a uniform thin film that may dry rapidly. Thus, the viscosities of compositions for use on intact skin of this disclosure may be no greater than 500,000 cps, no greater than 200,000 cps, no greater than 50,000 cps, no greater than 10,000 cps, or no greater than 5,000 cps. For use on skin, the composition may have a viscosity of less than 100 cps. For use on wounds or musocal tissue, such as in the nasal cavity or vagina, the viscosity may be relatively high to minimize drainage and mess but may be no greater than 20,000 cps. On wounds and mucosal tissue, the composition may not dry in use. Thus, the high viscosity helps to maintain the composition at the application site for extended periods of time to improve microbial kill.

This rapid and high antimicrobial activity is provided through the use of iodine, for example, delivered as an iodophor to reduce irritation potential, as the active antimicrobial agent. The compositions of the present disclosure may further comprise one or more hydroxycarboxylic acid buffers in particularly high use concentrations. The elevated concentrations of hydroxycarboxylic acids in the compositions contribute significantly to an increase in bacterial kill. By comparison, a composition of the present disclosure reduces normal skin flora by at least 0.5 log more than the same composition without the hydroxycarboxylic acid buffer present. This “same” composition includes additional water instead of the hydroxycarboxylic acid buffer and would be adjusted to the same pH as the composition with these components using a mineral acid or base, such as hydrochloric acid or sodium hydroxide that does not compromise the stability of the composition. The placebo compositions (i.e., compositions without an antimicrobial agent but still including the hydroxycarboxylic acid buffer) are relatively inactive. By comparison, a composition of the present disclosure reduces normal skin flora by at least 0.5 log more than the same composition without the iodine or iodophor present when tested on a dry human skin site (e.g., back or abdomen) according to ASTM testing method E1173-93 measured 2 minutes after completion of a 30-second scrub with gauze soaked in the composition using moderate pressure.

Generally, antiseptic compositions are applied to the tissue, typically skin, and allowed to dry and remain in place for at least 2 minutes, and often for several hours to days. Significantly, many of the compositions of the present disclosure maintain very low bacterial counts on the tissue, typically skin, for long periods of time, e.g., often up to 6 hours, and even up to 24 hours.

In some embodiments, the compositions of the present disclosure may be supplied in the ready to use concentration (i.e., can be directly applied to tissue). The compositions may also be prepared as concentrates that are diluted prior to use. For example, concentrates requiring dilution ratios of 0.5:1 to 3:1 parts water to concentrate are contemplated. The higher limit of the concentrate is limited by the solubility and compatibility of the various components at higher concentrations.

The compositions of the present disclosure may be applied to the skin or to mucosal surfaces (nares, urethra, vagina, etc.) using any suitable means. Ordinarily, an absorbent of some type such as gauze, foam sponges, non-woven fabrics, cotton fabrics, cotton swabs or balls, and the like, are soaked with the composition which is used to wipe the composition over the intended site. With very high activity compositions having exceptional wetting properties (e.g., higher alcohol content formulations), a single stroke prep may be all that is necessary. In most cases, however, it is believed that it helps to wipe the soaked absorbent across the skin several times, preferably in various directions, in order to thoroughly wet the skin and ensure good coverage into the finer details of the skin. In general, however, extensive scrubbing is not called for as is recommended by prior art products due to the enhanced activity resulting from the high concentration of organic buffer. For example, the manufacturer of BETADINE Surgical Scrub (Purdue Frederick Company, Norwalk, CT) specifies that the user scrub thoroughly for 5 minutes. The compositions of the present disclosure may require scrubbing for less than about 60 seconds, less than about 45 seconds, or less than about 30 seconds, followed by a 2-minute wait without blotting.

In some embodiments, it may be necessary to reduce microbial counts on the nares such that the reduction is maintained for at least 12 hours. In other cases, it may be necessary to add this repeatedly, twice a day or once a day for up to 21 days.

In order to maintain strict asepsis, the applier of a preoperative patient skin prep may start at the proposed site of the incision and work outward, thus preventing return to the incision site with a “dirty” applicator. Certain compositions of the present disclosure can be wiped on the skin in a simple overlapping motion taking care to cover each spot at least two or three times as the user works outward such that essentially no scrubbing is required.

### EXAMPLES

These examples are merely for illustrative purposes only and are not meant to be limiting on the scope of the appended claims. All parts, percentages, ratios, etc. in the examples and the rest of the specification are by weight, unless noted otherwise. Materials used in the examples and their sources are provided in Table 1. Solvents and other reagents used were obtained from the MilliporeSigma Company, St. Louis, MO, unless otherwise noted.

TABLE 1
Materials and vendors.

| Trade name | Material Description | Vendor (Location) |
|---|---|---|
| | PVP-Iodine | BASF (Florham Park, NJ) |
| | Hydroxypropyl cellulose | Aldrich (St. Louis, MO) |
| | Iota-Carrageenan | FMC Biopolymer (Philadelphia, PA) |
| | Carboxymethyl Cellulose Sodium | TCI Chemicals (Portland, OR) |
| Lauramine oxide | Lauramine oxide | Acme Hardesty (Blue Bell, PA) |
| Ammony X LMDO | Amido Type amine oxide | Stepan Company (Northfield, IL) |
| Crodafos SG | PPG-5-Cetcth-10-Phosphate | Croda, Inc. (Edison, NJ) |
| Ethoxcare 5011 | Ceteareth-25 | Ethox Chemicals (Greenville, SC) |
| Ethal SA 40 | Steareth-40 | Ethox Chemicals (Greenville, SC) |
| Hetoxyl STA 100 | Steareth 100 (Polyoxyethylene(100) | Global Seven (Columbus, OH) |
| | stearyl ether) | |
| Hipure 90 | L(+) Lactic Acid, USP | Purac America (Lincolnshire, IL) |
| | Malic Acid, NF (dl) | Lonza (Universal Preserve-A-Chem) |
| | | (Somerset, NJ) |
| | Xylitol, USP | Roquette America, Inc. (Keokuk, IA) |
| Celquat SC 230M | Polyquaternium-10 | National Starch (Bridgewater, NJ) |
| | Sodium Hydroxide, NF | J.T. Baker (Mallinckrodt Baker, Inc.) |
| | Sodium Iodide | Sigma Aldrich (St. Louis, MO) |
| | Purified Water, USP | Intermountain Life Sciences (Jordan, UT) |

### Test Methods

### Stability Data

Freeze-thaw and Centrifuge: Allegra 6R centrifuge from Beckman Coulter was used to perform centrifugation studies. All samples were tested at 1620 G-force for 10-15 minutes. Each composition was placed into a centrifuge tube and then centrifuged at ambient temperature, followed by freezing below −12° C. overnight, followed by thawing above 17° C. for a minimum of 4-5 hours. The freeze-thaw protocol was conducted two to three times prior to assessing freeze-thaw stability by conducting a viscosity measurement.

### Brookfield Viscosity Measurements

The viscosity of each composition was measured at approximately 22-24° C. at ambient pressure using a Brookfield LVDV-I+ viscometer equipped with a model D Brookfield heliopath and LV spindles. The spindle and speed were chosen for each sample such that the viscometer operated in the middle of its range. All samples were allowed to equilibrate at approximately 22-24° C. for 24 hours prior to measurement. The viscosity measurement was taken at the lowest spindle speed possible while maintaining the viscosity reading within 30-70% of the viscometer range. The viscosity measurements were done at 22-24° C. using 20 mL glass vials and S4 spindle size. At other times, vials were put into the oven at 52° C. for 4 days prior to measuring viscosity.

### Total Iodine Titration

Chemical stability was measured by iodine titration. Total iodine was determined by transferring about 3-4 g of accurately weighed (to the nearest 0.0001 g) composition to 100 mL polypropylene beakers. A magnetic stir bar, 50 mL of deionized water, and 12-15 drops of 6N sulfuric acid were added to each sample. The samples were stirred until dissolved. Titrations were then performed with 0.1000±0.0002 N sodium thiosulfate titrant using a Metrohm 751 automatic titrator and combination platinum electrode. The samples were run in duplicate. The average values of the total iodine measurements are recorded in Table 2.

Porcine mucosal model (PMM) efficacy assay protocol

### Tissue Preparation

Porcine vaginal mucosal tissue was scrubbed and rinsed with tap water. Then, 5-mm diameter biopsy explants were created, transferred into RPMI 1640 media+2% penicillin/streptomycin (P/S), trimmed, and sonicated for 2.0±0.1 minutes. Tissue explants were incubated for 15 minutes in RPMI 1640 media+2% P/S. Media was replaced with RPMI 1640 media with no antibiotics and washed 3 times. Explants were incubated in antibiotic free RPMI 1640 media for 15 minutes. Wells of a 6-well plate were filled with 2.0±0.1 mL RPMI 1640 media (no antibiotics, no fetal calf serum) and a transwell insert. The tissue explants were transferred to the transwells with epidermis side up (3 explants/well).

### Infection (MRSA Xen30)

A fresh petri plate was streaked directly from the bacterial test organism (MRSA Xen30) from frozen stock within one week of the experiment. A culture tube containing 5 mL Todd Hewitt (TH) broth was inoculated with multiple colonies and placed in a shaking incubator (37° C., 200 rpm) late in the afternoon on the day before the experiment. On the morning of the experiment, 20±5 microliters (μL) bacteria was passaged into 2 mL fresh TH broth and grown for 3±1 hours. The optical density of the culture was measured at 600 nm, for which a reading of 0.6 corresponds to ~5×10⁸ colony forming units (CFUs)/mL. Dilutions were prepared in RPMI 1640 media such that 1 mL contains ~5×10⁸ CFUs. Bacteria (2±1 μL) were transferred to each explant and the explants were returned to incubator to infect for 2±0.25 hours.

### Treatment

A fixed volume (100 μL) of each treatment solution (at pH 3.2) was applied to the tissue to provide an excess of treatment solution on tissue. The treated bacteria-inoculated explants were incubated at 37±2° C. for 1 hour and then were rinsed gently with 1 mL 2% mucin solution (using a repeat pipettor, dispensing 330 μL per explant at a set injection speed). The mucin solution was aspirated from around each explant and replaced with fresh media. The explants were incubated at 37±2° C. for 1 hour and 24 hours.

### Sampling/Counting

The explants were removed from the transwells and placed into microcentrifuge tubes containing 1 mL 2×DE broth neutralizer. Bacteria were liberated using a vortex/sonicate/vortex protocol (30±5 seconds vortex, 2±0.5 minutes sonicate, 30±5 seconds vortex). Bacteria were plated (using a spiral plater) onto MSA plates (diluted in PBS if necessary). Plates were incubated overnight at 37° C. with ambient CO₂. Testing of the samples was done in triplicate, and data are presented as mean log recovery of bacteria±standard deviation.

### Example 1: Formulations

Formulations were prepared according to Table 2. In a typical experiment, water to total 100 wt % of each formulation was weighed into a vial, then Povidone Iodine, lactic acid and non-ionic surfactant were added sequentially and stirred with a magnetic stirrer or mixed using a blade mixer. After 30 minutes of mixing, Xylitol, sodium iodide and sodium hydroxide were added and mixed. Finally, celquat was added and the formulation was allowed to thicken for at least 30 minutes with stirring by a blade mixer or stirred overnight with a magnetic stirrer. Sample pH was measured and adjusted if necessary to 3.1-3.4.

### Example 2: Physical Stability of Compositions

Physical stability was assessed by conducting freeze-thaw experiments for each formulation according to the test method described earlier. In the example, all of the compositions contain PVP-I and lactic acid and sodium iodide as well as xylitol and Celquat. Composition 1 shows that compositions containing PVP-I and lactic acid can be stabilized by relatively high concentration of nonionic surfactant (Tables 2 and 3). Using a relatively lower concentration of nonionic surfactant makes composition 2 and 3 unstable, The results for composition 5 and 6 show that the zwitterionic surfactant results in good stability.

Composition 7 was stabilized with steareth 100 with half the level of Povidone Iodine. The solution was stabilized with lower levels of sodium iodide. Compositions 8, 9 and 10 were not physically stable with steareth 100.

Thus, the examples show the power of using nonionic surfactants in stabilizing antiseptic compositions comprising PVP-I and lactic acid. The use of relatively safe nonionic surfactants in stabilizing compositions of PVP-I comprising hydroxycarboxylic acid like lactic acid should improve the safety of use of such compositions on sensitive tissues.

TABLE 2
Formulation compositions and physical stability of formulations containing non-ionic surfactants. Numerical
values are percentages by weight of each component in samples and the remaining weight percent is water.

| Formulations | | #1 | #2 | #3 | #4 | #5 | #6 | #7 | #8 | #9 | #10 | #11 |
|---|---|---|---|---|---|---|---|---|---|---|---|---|
| Components | | | | | | | | | | | | |
| Povidone Iodine | | 5 | 5 | 5 | 5 | 5 | 5 | 2.5 | 5 | 5 | 5 | 5 |
| Lactic Acid | | 5 | 5 | 5 | 5 | 5 | 5 | 5 | 5 | 5 | 5 | 5 |
| Malic acid | | | | | | | | | | | | 2 |
| Ammonyx LMDO | | | | | | | | | | | | 0.75 |
| (30% solution | | | | | | | | | | | | |
| in water) | | | | | | | | | | | | |
| Nonionic | Ceteareth | 2 | 0.7 | 1 | 1.4 | 1.4 | 1.4 | 0 | 0 | 0 | | 0 |
| surfac- | 25 | | | | | | | | | | | |
| tant | Steareth | | | | 0 | | 0 | 1.4 | 2 | 3 | 4 | 1.4 |
| | 100 | | | | | | | | | | | |

| | | | | | | | | | | | |
|---|---|---|---|---|---|---|---|---|---|---|---|
| Lauramine oxide | 0 | 0 | 0 | 0 | 0.25 | 0.75 | 0 | 0 | 0 | 0 | |
| (30% solution | | | | | | | | | | | |
| in water) | | | | | | | | | | | |
| Cationic thickener | 1 | 1 | 1 | 1 | 1 | 1 | 1 | 1 | 1 | 1 | 1 |
| Celquat SC-230M | | | | | | | | | | | |
| xylitol | 10 | 10 | 10 | 10 | 10 | 10 | 10 | 10 | 10 | 10 | 10 |
| Crodafos SG | | | | | | | | | | | 1 |
| Sodium Iodide | 2.5 | 2.5 | 2.5 | 2.5 | 2.5 | 2.5 | 1.5 | 2.5 | 2.5 | 2.5 | 2.5 |
| NaOH | 0.6 | 0.6 | 0.6 | 0.6 | 0.6 | 0.6 | 0.6 | 0.6 | 0.6 | 0.6 | 0.6 |
| Properties | | | | | | | | | | | |
| Viscosity (cp) | 4160 (3600) | 3780 | 3260 | 4020 | 4220 (3400) | 4740 (4180) | 3360 (2840) | | | | |
| Initial (52° | | | | | | | | | | | |
| C. 4 days) | | | | | | | | | | | |
| Chemical stability | 9.96% | | | | 0.68% | 1.02% | | | | | |
| (% loss 60 | | | | | | | | | | | |
| C., 1 wk) | | | | | | | | | | | |
| Chemical stability | 4.5% | | | | 2.77% | 1.1% | 10.25% | | | | |
| (% loss 50 | | | | | | | | | | | |
| C., 1 wk) | | | | | | | | | | | |
| Chemical stability | 5.0% | | | | 0% | 6.2% | 9.1% | | | | |
| (% loss 50° | | | | | | | | | | | |
| C., 8 wk) | | | | | | | | | | | |
| Physical stability | Sta- | Sta- | Sta- | Sta- | Sta- | Sta- | Sta- | Unsta- | Unsta- | Unsta- | |
| (freeze-thaw) | ble | ble | ble | ble | ble | ble | ble | ble | ble | ble | |

TABLE 3
Non-ionic surfactants.

| | Hydrophobic- | Hydroxyl |
|---|---|---|
| | Lipophilic | value |
| Surfactant type | Balance | (mg KOH/g) |
| Steareth-100 (Hetoxyl STA 100) | 18.8 | 12.7 |
| Steareth-40 (Ethal SA 40) | 16.6-17.8 | 28.4 |
| Ceteareth-25 (C-25) | 16.25 | 24 |

At the higher Povidone Iodine concentrations (5%), stable formulations were obtained with ceteareth-25 in the absence of anionic surfactant. Addition of lauramine oxide helps with increased chemical stability for the ceteareth-25 formulations. Steareth-100 did not appear to have good physical stability at the higher Povidone Iodine formulations. However, Steareth 100 appeared to stabilize formulations at lower levels of Povidone Iodine.

### Example 3: Addition of Thickeners for Stability of Formulations Containing Povidone Iodine and Monofunctional Acid

Formulations were prepared according to Table 4 using the methods described above and their properties were evaluated as described above.

TABLE 4
Formulations.

| Formulations | #12 | #13 | #14 |
|---|---|---|---|
| Component (wt. %) | | | |
| Water | To total 100 | To total 100 | To total 100 |
| Lactic Acid | 5 | 5 | 5 |
| Surfactant (Ceteareth 25) | 2 | 2 | 2 |
| Thickener | NaCMC 2% | Carrageenan 2% | Hydroxypropyl |
| | | | cellulose 2% |
| NaI | 2.5 | 2.5 | 2.5 |
| xylitol | 10 | 10 | 10 |
| Sodium Hydroxide | 0.6 | 0.6 | 0.6 |
| Povidone Iodine | 5 | 5 | 5 |
| Total | 100 | 100 | 100 |
| Properties | | | |
| Final pH 3.2 | 3.12 | 3.1 | 3.1 |
| Chemical stability (60° C. 1 week) | >20% loss | >20% loss | >20% loss |
| Viscosity | 6210 cP | 1720 cP | 670 cP |
| Physical stability | Not stable | Not stable | Not stable |

The data from Table 4 demonstrates that the non-ionic and the anionic thickeners are not as effective as the cationic thickener in building viscosity and keeping the formulation stable.

### Example 4: Efficacy of the 2.5% Povidone Iodine Formula Versus the 5% Povidone Iodine Formula and Persistence

No detectable growth was observed with one treatment of formulas 1, 5, 6 and 7 in Table 2 with an inoculum of 5.42 Log₁₀ CFU/Explant after 1 hour, whereas betadine (10% Povidone Iodine solution) had close to 7 logs in recovery while formulas 1, 5, 6 and 7 continued to have no recovery at the 24 hour time point.

So, the 5% formula had similar activity to 2.5% Povidone Iodine for short and long periods of time (at 1 hour and 24 hours).

### Example 5

PMM MTT viability assay: (Test to mimic more than one application of formulation)

To determine the viability of formulations:

Viability

Viability in explant tissue model with repeat dose.

$$RPMI 1640 = Rosewell Park Memorial Institute Medium$$

$$ABX = 2 \% penicillin / streptomycin$$

$$FCS = fetal calf serum$$

### A. Tissue Prep:

Porcine Mucosal Model (PMM) tissue section was scrubbed and rinsed with tap water. Five millimeter diameter biopsy explants were created, transferred into RPMI 1640±2% ABX, trimmed, and washed three times in RPMI without antibiotics. Explants were incubated for at least 30 min (ABX washout). To each well of a 6-well plate was added 2.0±0.1 mL RPMI (no ABX, no FCS) per well and transwell insert was added. Tissue explants were transferred lumen side up to the insert (3 explants/well).

### B. Treatment:

Formulations 1, 5, 6, 7, and 11 (Table 2) were evaluated. Ten microliters of formulation was applied per explant. Explants were incubated at 37±2° C. for 1 h and then rinsed gently with 1 mL of 2% mucin solution per well. For each explant, 330 μL of 2% mucin was dispensed using a repeat pipettor at an ejection speed of 4. Mucin was aspirated from around each explant. Media was aspirated and replaced with fresh media under the transwell. Explants were incubated at 37±2° C. for 24 h. 10 μL of formulation was applied again to each explant, and explants were washed with mucin after 1 h. Mucin was aspirated, and media was replaced as above. Explants were incubated at 37±2° C. for 24 h.

### C. MTT Assay

Explants were washed by swirling in 200±20 μL PBS three times (three separate wells, each containing 200±20 μL PBS). Explants were transferred to a 96 well plate with 100±10 μL RPMI & 10±1 μL MTT substrate per well. The explants were incubated in the plate submerged in the MTT substrate at 37° C.+7% CO₂ for 3 h. The explants were transferred to a 96 well plate containing 100±10 μL de-staining solution per well and sealed with plate film (one explant per well). The sealed plate was incubated at 4° C. overnight. Explants were removed from wells. Plate was read at 570 nm and 690 nm and percent viability was calculated using the following formula: % viability=100×[OD(sample)/OD(negative control)]

Results: Formulations 1, 5, 6 and 7 in Table 2 each had greater than 30% viability while formulation 11 had a viability of less than 30% after two applications and measured at the 48 hour time point.

1. An antiseptic composition comprising:
an antimicrobial agent comprising molecular iodine (I₂),
an iodide salt;
a hydroxycarboxylic acid present at a concentration of at least 2 wt-%, based on the total weight of the antiseptic composition; and
a nonionic surfactant;
wherein the antiseptic composition comprises no amount of anionic surfactant.

2. The antiseptic composition of claim 1, wherein the hydroxycarboxylic acid comprises a monocarboxylic acid.

3. The antiseptic composition of claim 1, wherein the nonionic surfactant has an HLB value of between 12 and 20.

4. The antiseptic composition of claim 1, wherein any surfactants present in the antiseptic composition are included in the U.S. FDA list of approved inactive excipients.

5. The antiseptic composition of claim 1, further comprising a zwitterionic surfactant.

6. The antiseptic composition of claim 1, wherein the composition is iodine stable.

7. The antiseptic composition of claim 1, wherein the composition is physically stable.

8. The antiseptic composition of claim 1, wherein the hydroxycarboxylic acid is present in an amount of between 2 wt-% and 10 wt-%, based on the total weight of the antiseptic composition.

9. The antiseptic composition of claim 1, further comprising an amine oxide.

10. The antiseptic composition of claim 8, wherein the amine oxide is present in a concentration of 0.25 wt-% to 1.5 wt-%, based on the total weight of the antiseptic composition.

11. The antiseptic composition of claim 1, wherein the iodide salt is present at an amount of between 0.25 wt-% and 5 wt-%, based on the total weight of the antiseptic composition.

12. The antiseptic composition of claim 1, wherein the antimicrobial agent comprises an iodophor.

13. The antiseptic composition of claim 12, wherein the iodophor is povidone-iodine.

14. The antiseptic composition of claim 13, wherein the povidone-iodine concentration is greater than 3 wt. %, based on the total weight to the composition, and wherein the non-ionic surfactant has an HLB value of between 12 and 17 and a hydroxyl value (mg KOH/g)) of between 35 and 45.

15. The antiseptic composition of claim 13, wherein the povidone-iodine concentration is less than 3 wt. %, based on the total weight to the composition, and wherein the non-ionic surfactant has HLB value of between 18 and 20 and a hydroxyl value (mg KOH/g)) of between 12 and 20.

16. The antiseptic composition of claim 1, further comprising a monosaccharide, a sugar alcohol, or a combination thereof.

17. The antiseptic composition of claim 16, wherein the monosaccharide, sugar alcohol, or combination thereof is present at a concentration of greater than 5 wt-%, based on the total weight of the antiseptic composition.

18. The antiseptic composition of claim 1, wherein the total iodine level in the composition is less than 2.7 wt-%, based on the total weight of the antiseptic composition

19. The antiseptic composition of claim 1, further comprising a thickener comprising a cationic polymer.

20. A method of decolonizing or disinfecting the nasal passages of a subject, the method comprising applying the composition of claim 1 to the nasal passages of the subject.

21. A method of disinfecting the tissue of a subject, the method comprising applying the composition of claim 1 to the nasal passages of the subject.

22. A method of forming a stable an antiseptic composition comprising an iodophor and a hydroxycarboxylic acid, the method comprising:
providing an antimicrobial agent comprising molecular iodine (I₂),
providing a hydroxycarboxylic acid;
providing an iodide salt; and
providing a nonionic surfactant; and
mixing the antimicrobial agent, hydroxycarboxylic acid; iodide salt, and nonionic surfactant to form a stabilized composition;
wherein the antiseptic composition comprises no amount of anionic surfactant.

23. An antiseptic composition comprising:
an antimicrobial agent comprising an iodophor,
an iodide salt;
a hydroxycarboxylic acid present at a concentration of at least 2 wt-%, based on the total weight of the antiseptic composition; and
a nonionic surfactant in an amount of at least 1 wt-%, based on the total weight of the antiseptic composition, wherein the nonionic surfactant has an HLB value of between 12 and 20.
